(19) United States
(12) Patent Application Publication
(10) Pub. No.: US 2023/0346652 A1
(43) Pub. Date: Nov. 2, 2023

(54) Foaming Hand Sanitizer with High Alcohol Content

(71) Applicant: Ele' Corporation, McCook, IL (US)

(72) Inventors: Severine JEULIN, Chicago, IL (US); Enchelle THOMAS, Blue Island, IL (US); Alexander PAPADAKIS, Oak Park, IL (US)

(73) Assignee: Ele' Corporation, McCook, IL (US)

(21) Appl. No.: 18/220,101

(22) Filed: Jul. 10, 2023

Related U.S. Application Data
(63) Continuation of application No. PCT/US2022/011271, filed on Jan. 5, 2022.
(60) Provisional application No. 63/136,893, filed on Jan. 13, 2021.

(51) Int. Cl. A61K 8/04; A61K 8/34; A61K 8/894; A61Q 17/00

(52) U.S. Cl. CPC A61K 8/046; A61K 8/34; A61K 8/894; A61Q 17/005; A61K 2800/74

(57) ABSTRACT

Embodiments of the present invention provide a foaming alcohol-based hand sanitizer having good foaming and virucidal characteristics by combining a linear siloxane polyether having a siloxane backbone with between 20 and 40 repeating siloxane units, two polyethylene glycol backbones each having between 10 and 20 repeating ethylene oxide units, and two polypropylene glycol backbones each having between 0 and 10 propylene oxide units, an alcohol such as ethanol or isopropanol, and water, where the hand sanitizer comprises between 0.01% and 5% linear siloxane polyether, more than 85% alcohol, and the balance is water.

### PRIORITY

This application claims the benefit of U.S. App. No. 63/136,893 titled “Foaming Hand Sanitizer with High Alcohol Content” which was filed on Jan. 13, 2021.

### TECHNICAL FIELD

The present invention relates to foaming hand sanitizers which utilize a linear polysiloxane polyether as the foaming agent and an alcohol as the virucidal agent.

### BACKGROUND ART

Viruses cause about 5% of all healthcare associated infections, and the use of ethanol-based hand rubs can prevent such infections. However, the efficacy of ethanol-based hand rubs with respect to various types of viruses depends on the concentration of ethanol. By way of example, ethanol concentrations of less than 75% (by weight) were effective within 30 seconds against enveloped viruses, however, a concentration of up to 95% was necessary to kill certain non-enveloped viruses within 30 seconds. See, e.g., Kampf, Guenter, Efficacy of Ethanol against Viruses in Hand Disinfection, JOURNAL OF HOSPITAL INFECTION 98, pp. 331-338 (2018).

Ethanol-based hand rubs can be dispensed as liquids, gels, and foams. For a number of usability reasons, including fast absorption, reduced drying time, low smell, gentleness to the skin, clean/non-sticky feel, and dosage control, foam-based hand sanitizers are viewed as superior to liquid- and gel-based hand sanitizers. See, e.g., Greenaway, R. E. et al., Impact of Hand Sanitizer Format (Gel/Foam/Liquid) and Dose Amount on its Sensory Properties and Acceptability for Improving Hand Hygiene Compliance, JOURNAL OF HOSPITAL INFECTION 100, pp. 195-201 (2018). The foam may be dispensed using propellant and non-propellant foam dispensing devices known in the art.

Alcohol-based foaming hand sanitizers known in the art typically include an alcohol such as ethanol or isopropanol, water, and a linear polysiloxane polyether foaming agent. For example, U.S. Pat. No. 8,058,315 discloses the combination of an alcohol (ethanol, propanols, or butanols), water, and a polyethylene glycol (PEG) linear dimethicone surfactant with a length of PEG-8, PEG-10, or PEG-12. Similarly, U.S. Pat. Pub. No. 20150297728 discloses the combination of ethanol, water, and a linear dimethicone copolyol with a polyethylene glycol of PEG-8, PEG-10, or PEG-12. However, the inventors of the present invention have experimentally determined that the foams based on such prior art formulations do not exhibit high foaming quantity, quality, and stability when the alcohol content exceeds 85% by weight; in particular, less foam is produced, the foam breaks down more quickly, the foam is less dense, and the air bubbles are bigger and non-uniform in size.

Therefore, what is needed is a foaming hand sanitizer that has an alcohol content sufficient to kill most viruses within 30 seconds that still provides high foam quantity, quality, and stability comparable to foaming hand sanitizers having a lower alcohol content.

### SUMMARY OF INVENTION

Embodiments of the present invention relate to formulation of a foaming alcohol-based hand sanitizer that (a) has an alcohol content high enough to kill most viruses within 30 seconds and (b) has foam properties of comparable quantity, quality, and stability to that of lower alcohol content foaming hand sanitizers. More particularly, the embodiments of the present invention utilize a linear polysiloxane polyether having a polysiloxane backbone with between 20 and 40 repeating dimethyl siloxane units and blocked by propyl segments, polyethyl ether chains (each having between 10 and 20 repeating ethyl ether units), polypropyl ether chains (each having between 0 and 10 repeating propylene oxide units), and terminal hydroxyls. Embodiments of the present invention comprise between 0.01 and 5% by weight of the linear polysiloxane polyether, at least 85% alcohol by weight, and the remainder being water.

### BRIEF DESCRIPTION OF THE DRAWINGS

FIG. 1 is a line drawing of an embodiment of a polypropyl ether/polyethyl ether foaming agent utilized in the present invention.

FIG. 2 is a line drawing of an embodiment of a polyethyl ether foaming agent utilized in the present invention.

FIGS. 3a through 3i are photographs showing foam quality appearances for foam quality scores between 1 and 5.

FIG. 4 is a table summarizing foam quantity and foam quality for prior art compositions and for compositions prepared according to the present invention.

### DETAILED DESCRIPTION OF THE INVENTION

In the following embodiments of the present invention, polymer segments are expressed in terms of the number of repeating monomer units. These numbers should be interpreted as referring to the statistical average number of monomer units in each molecule and not an exact numerical requirement.

Embodiments of the present invention utilize a linear polysiloxane polyether comprising a polysiloxane segment having between 20 and 40 —OSi(CH₃)₂— units, blocked on each side by a propyl segment, a polyethyl ether segment having between 10 and 20 —CH₂CH₂O— units, an optional polypropyl ether segment having between 1 and 10 —CHCH₃CH₂O— units and a terminating hydroxy.

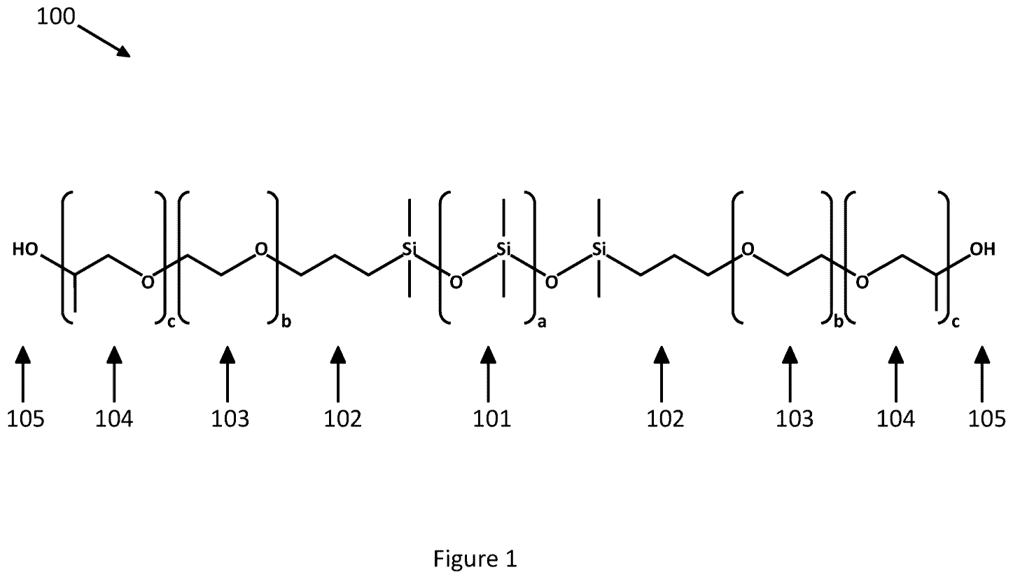

FIG. 1 depicts linear polysiloxane polyether 100 which comprises dimethyl siloxane backbone 101 having between 20 and 40 repeating —OSi(CH₃)₂— units (that is, the “a” subscript is between 20 and 40). Dimethyl siloxane backbone 101 is blocked on each side by a propyl (C₃H₆) segment 102, a polyethyl segment 103 having between 10 and 20 repeating —OCH₂CH₂— units (that is, the “b” subscript is between 10 and 20), a polypropyl ether segment 104 having between 1 and 10 repeating —CHCH₃CH₂O— units (that is, the “c” subscript is between 1 and 10), and a terminating hydroxy 105.

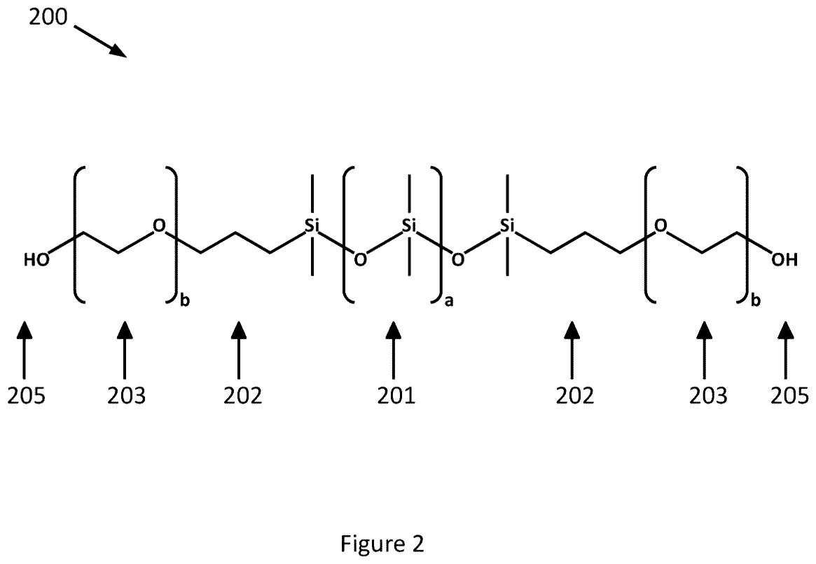

FIG. 2 depicts linear polysiloxane polyether 200 which comprises dimethyl siloxane backbone 201 having between 20 and 40 repeating —OSi(CH₃)₂— units (that is, the “a” subscript is between 20 and 40). Dimethyl siloxane backbone 201 is blocked on each side by a propyl (C₃H₆) segment 202, a polyethyl segment 203 having between 10 and 20 repeating —OCH₂CH₂— units (that is, the “b” subscript is between 10 and 20), and a terminating hydroxy 205.

The foregoing linear polysiloxane polyethers 100 and 200 are combined with ethanol and water to form the inventive foaming hand sanitizer, where the foaming hand sanitizer contains at least 85% ethanol, between 0.01% and 5% linear polysiloxane polyether, with the balance of foaming hand sanitizer being water (all percentages measured by weight).

In a preferred embodiment utilizing linear polysiloxane polyether 200, the inventive foaming hand sanitizer comprises:
1.75% linear polysiloxane polyether where a=36 and b=12,
90% ethanol, and
8.25% water,
where all percentages are measured by weight.

### Evaluation of Foaming Characteristics

In order to evaluate the foaming characteristics of foaming hand sanitizers, the inventors have adapted techniques known in the art for measuring foam quality and quantity. By comparing various formulations of foaming hand sanitizers under controlled procedures, the inventors have identified which formulations produce the highest quality and quantity of foam.

To measure foam quantity, the inventors poured a 50 g sample of the subject foam hand sanitizer into a woo-watt Oster® Classic Series Heritage Blender, blended the sample at the highest speed for 10 seconds, poured the sample into a 100 ml graduated cylinder, and measured the total volume of the foam and any un-foamed liquid immediately, after 30 seconds, and after 5 minutes.

To measure foam quality, the inventors placed a sample of the subject foam hand sanitizer into a 250 ml PET bottle with a foamer pump, commercially available from SKS Bottle & Packaging Inc. (item #0043-12), priming the foaming pump head with several pumps, performing one additional pump onto a flat, black, circular plastic surface 58 mm in diameter, and then photographing the surface immediately and again after 30 seconds. Three panelists independently graded the foam quality on a 1 to 5 scale defined as follows:

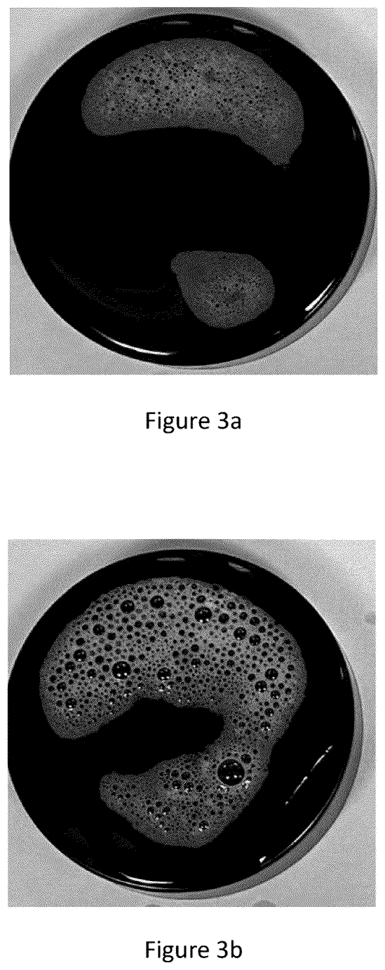

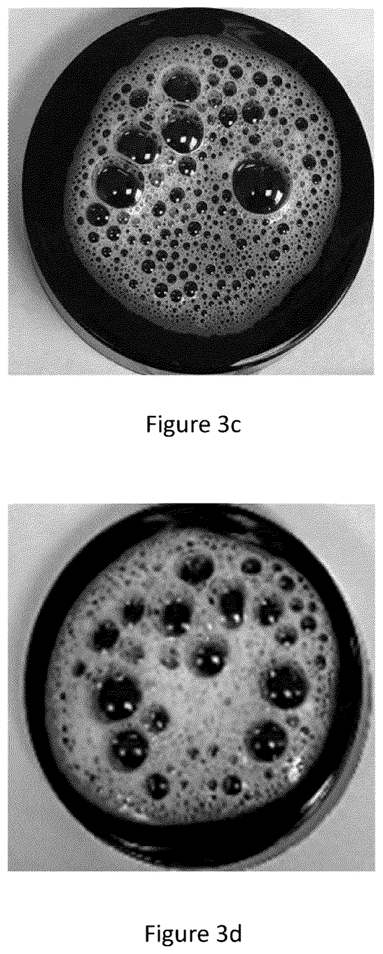

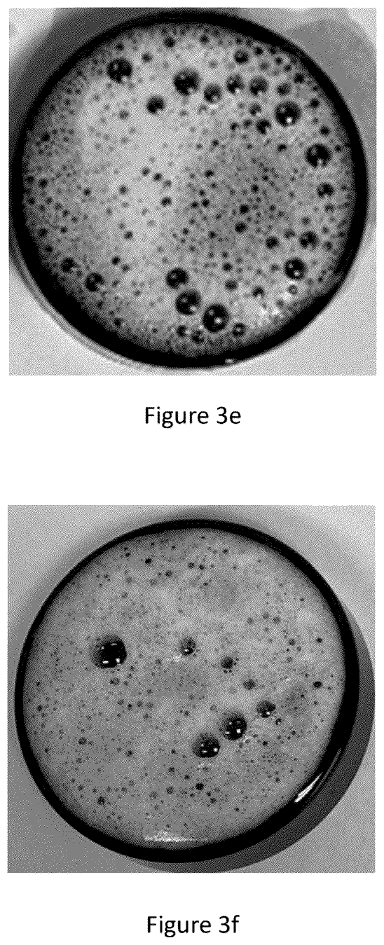

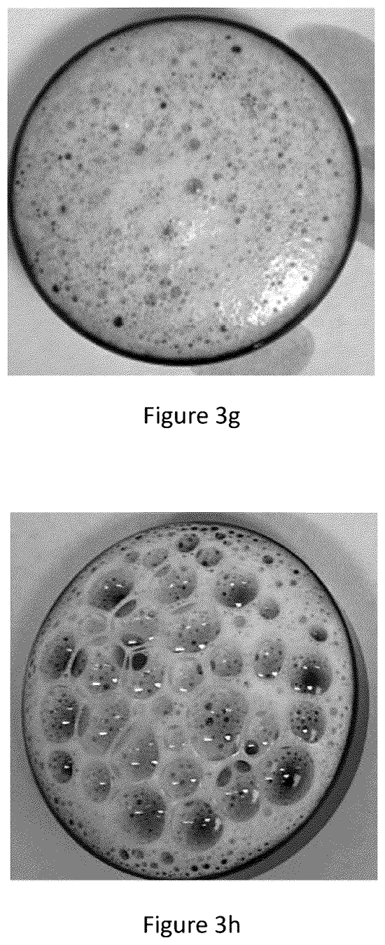

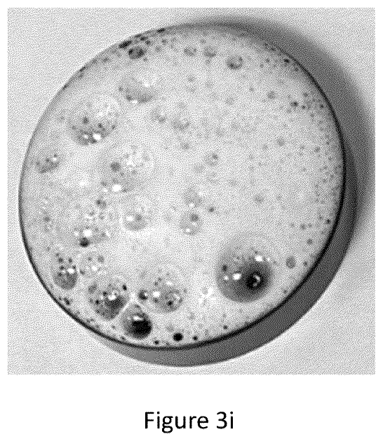

1.0 (poor). This foam exhibits few to no visible bubbles of random sizes, and the black surface area is completely visible through the liquid/foam. See FIG. 3a.

1.5 (poor to low). This foam exhibits few visible bubbles of small sizes, and the black surface area is nearly completely visible through the foam. See FIG. 3b.

2.0 (low). This foam exhibits loosely-packed bubbles of random sizes, and less than half of the black surface area is covered with foam. See FIG. 3c.

2.5 (low to average). This foam exhibits low density, random-sized bubbles, and about half of the black surface area is covered with foam. See FIG. 3d.

3.0 (average). This foam exhibits loose-to-dense, large, and tightly-packed bubbles, and more than half of black surface area is covered with foam. See FIG. 3e.

3.5 (average to good). This foam exhibits mostly small, homogenous-sized bubbles size with a few larger bubbles and little to no volume, and nearly all of the black surface area is covered with foam. See FIG. 3f.

4.0 (good). This foam exhibits loose to dense, nearly same-sized, tightly packed bubbles, with low volume, and the black surface area is completely covered with foam. See FIG. 3g.

4.5 (good to excellent). This foam exhibits loose to dense, lacy bubbles in high volume, and the black surface area is completely covered with foam. See FIG. 3h.

5.0 (excellent). This foam exhibits densely-packed, mostly or completely same-sized bubbles, in high volume, and the black surface is mostly or completely covered with foam. See FIG. 3i.

Based on the inventors' empirical observations using these qualitative and quantitative techniques, compositions having an initial foam volume of at least 95 ml have the best potential to generate foams with enough quantity, quality, and stability when dispensed with non-propellent foam packaging. Further, a foam quality score of at least 3.5 is needed to fully cover the consumer's hand with foam, and a foam quality score of 4.5 to 5.0 provides higher spreadability and hand coverage, reducing the occurrences of un-foamed liquid dripping through the consumer's fingers upon application.

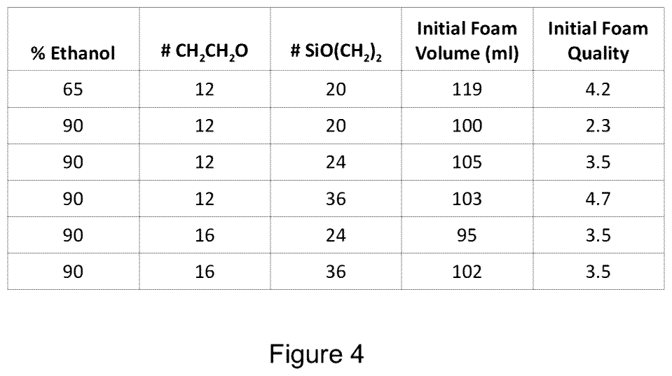

To evaluate the performance of the embodiments of the present invention using these qualitative and quantitative techniques, the inventors first established a baseline using a prior art linear polyethyl polysiloxane surfactant comprising 20 dimethyl siloxane units and two terminal polyethylene glycol segments, each having 12 ethyl ether units. This prior art linear polyethyl polysiloxane is commercially-available from Ele′ Corporation under the brand name PEL-SIL™ Bis-PEG-12. The inventors tested two foaming hand sanitizer formulations using this prior art surfactant, one with 65% ethanol and the other with 90%; both formulations used 1.75% prior art surfactant with the balance being water. As shown by the table in FIG. 4, the first formulation produced foam of acceptable quality and quantity, but its lower ethanol concentration limited its virucidal effect. The second formulation, while having a sufficient ethanol concentration to provide the requisite virucidal effects, failed to produce foam of acceptable quality and quantity.

Next, the inventors prepared hand sanitizer formulations using embodiments of the linear polyethyl polysiloxane surfactant of the present invention; all formulations had 90% ethanol, 1.75% surfactant, and 8.25% water (all percentages measured by weight). As can be seen in from the table in FIG. 4, these formulations produced high quality foams, which unlike the prior art formulation, had a sufficient ethanol concentration to exhibit high virucidal properties.

### Additional Embodiments

The foaming hand sanitizers described in the foregoing embodiments of the present invention may be modified and/or extended by one of ordinary skill without departing from the spirit of the present invention, so long as (a) the virucidal efficacy after 30 seconds achieves≥log₁₀ reduction of viral infectivity in suspension tests or the limit of detection is reached, (b) the foam quality using the foregoing techniques indicated a foam quality score ≥3.5, and (c) the foam quantity using the foregoing techniques indicated a foam volume of 95 ml. Selection of some of these modifications and extensions may affect the quality of the general characteristics of the present invention in terms of performance when used for particular applications. Other modifications and extensions may be driven by costs of manufacture, availability of materials, physical constraints, and other factors which may be independent of the general characteristics of the present invention. The following variations represent a non-exclusive list of examples of other embodiments which may be mixed and matched as needed and as technically feasible without affecting the general characteristics of the present invention.

In the foregoing foaming hand sanitizer embodiments, ethanol acts as the virucidal agent. In other embodiments, methanol, isopropanol, butanol, or other water-miscible alcohols may be used, either alone or in combination.

In the foregoing foaming hand sanitizer embodiments, the polysiloxane backbone is separated from the propyl ether backbone by a linear propyl segment. In other embodiments, this segment could be non-linear and it could include a different number of carbons (typically between 1 and 5).

In the foregoing foaming hand sanitizer embodiments, the disclosed formulation comprises ethanol, water, and the inventive linear polysiloxane polyether. In other embodiments, the foaming hand sanitizer may include additional components typically found in foaming hand sanitizer products, including additional surfactants, foam stabilizers, fragrances, coloring agents, terpenoids, humectants, skin conditioning agents, chelating agents, preservatives, thickeners, skin feel enhancers, and antioxidants. Such additional components would typically take the place of a portion of the water.

The procedures used to evaluate the quality and quantity of foam for any given formulation of the foaming hand sanitizer in comparison to prior art foaming hand sanitizers may be modified as needed, provided that the same procedures and scales are used for all samples in the comparison.

In the embodiment shown in FIG. 2, the polyethyl ether and polypropyl ether segments are distinct blocks. In other embodiments, the ethyl ether and propyl ether units could be interspersed.

1. A foaming hand sanitizer, comprising:
a linear polysiloxane polyether;
an alcohol; and
water,
where the linear polysiloxane polyether makes up between 0.01 and 5% of the weight of the foaming hand sanitizer and the alcohol makes up at least 90% of the weight of the foaming hand sanitizer, and
where the linear polysiloxane polyether comprises a polysiloxane backbone having between 24 and 40 repeating dimethyl siloxane units blocked on each side by an alkyl having between 1 and 5 carbons, a polyethyl ether chain having between 10 and 20 repeating ethyl ether units, and a terminating hydroxyl.

2. The foaming hand sanitizer of claim 1,
where the alcohol is ethanol.

3. The foaming hand sanitizer of claim 1,
where the alcohol is isopropanol.

4. The foaming hand sanitizer of claim 1,
where the polysiloxane backbone has 24 repeating dimethyl siloxane units and
where the polyethyl ether chain has 12 repeating ethyl ether units.

5. The foaming hand sanitizer of claim 1,
where the polysiloxane backbone has 24 repeating dimethyl siloxane units and
where the polyethyl ether chain has 16 repeating ethyl ether units.

6. The foaming hand sanitizer of claim 1,
where the polysiloxane backbone has 36 repeating dimethyl siloxane units and
where the polyethyl ether chain has 12 repeating ethyl ether units.

7. The foaming hand sanitizer of claim 1,
where the polysiloxane backbone has 36 repeating dimethyl siloxane units and
where the polyethyl ether chain has 16 repeating ethyl ether units.

8. The foaming hand sanitizer of claim 1,
where the alkyl is propyl.

9. The foaming hand sanitizer of claim 1,
where the linear polysiloxane polyether makes up about 1.75% of the weight of the foaming hand sanitizer, the alcohol makes up about 90% of the weight of the foaming hand sanitizer, and the water makes up about 8.25% of the weight of the foaming hand sanitizer.

10. The foaming hand sanitizer of claim 1, further comprising:
an additional surfactant, a foam stabilizer, a fragrance, a coloring agent, a terpenoid, a humectant, a skin conditioning agent, a chelating agent, a preservative, a thickener, a skin feel enhancer, or an antioxidant.

11. The foaming hand sanitizer of claim 1,
where the linear polysiloxane polyether is further blocked by two polypropyl ether chains having between 1 and 10 repeating propyl ether units and
where each polypropyl ether chain is positioned between one of the polyethyl ether chains and its nearest terminal hydroxyl.

12. The foaming hand sanitizer of claim 1,
where the linear polysiloxane polyether further comprises between 1 and 10 propyl ether units interspersed in each of the polyethyl ether chains.

13. A foaming hand sanitizer, comprising:
a linear polysiloxane polyether;
ethanol; and
water,
where the linear polysiloxane polyether makes up about 1.75% of the weight of the foaming hand sanitizer and the ethanol makes up about 90% of the weight of the foaming hand sanitizer,
where the linear polysiloxane polyether comprises a polysiloxane backbone having 24 repeating dimethyl siloxane units blocked on each side by propyl, a polyethyl ether chain having 12 repeating ethyl ether units, and a terminating hydroxyl.

14. A foaming hand sanitizer, comprising:
a linear polysiloxane polyether;
ethanol; and
water,
where the linear polysiloxane polyether makes up about 1.75% of the weight of the foaming hand sanitizer and the ethanol makes up about 90% of the weight of the foaming hand sanitizer,
where the linear polysiloxane polyether comprises a polysiloxane backbone having 24 repeating dimethyl siloxane units blocked on each side by propyl, a polyethyl ether chain having 16 repeating ethyl ether units, and a terminating hydroxyl.

15. A foaming hand sanitizer, comprising:
a linear polysiloxane polyether;
ethanol; and
water,
where the linear polysiloxane polyether makes up about 1.75% of the weight of the foaming hand sanitizer and the ethanol makes up about 90% of the weight of the foaming hand sanitizer,
where the linear polysiloxane polyether comprises a polysiloxane backbone having 36 repeating dimethyl siloxane units blocked on each side by propyl, a polyethyl ether chain having 12 repeating ethyl ether units, and a terminating hydroxyl.

16. A foaming hand sanitizer, comprising:
a linear polysiloxane polyether;
ethanol; and
water,
where the linear polysiloxane polyether makes up about 1.75% of the weight of the foaming hand sanitizer and the ethanol makes up about 90% of the weight of the foaming hand sanitizer,
where the linear polysiloxane polyether comprises a polysiloxane backbone having 36 repeating dimethyl siloxane units blocked on each side by propyl, a polyethyl ether chain having 16 repeating ethyl ether units, and a terminating hydroxyl.

17. A linear polysiloxane polyether surfactant, comprising:
a polysiloxane backbone having between 24 and 40 repeating dimethyl siloxane units;
an alkyl having between 1 and 5 carbons;
a polyethyl ether chain having between 10 and 20 repeating ethyl ether units; and
a terminating hydroxyl.

18. The linear polysiloxane polyether surfactant of claim 17, comprising:
where the polysiloxane backbone has 24 repeating dimethyl siloxane units and
where the polyethyl ether chain has 12 repeating ethyl ether units.

19. The linear polysiloxane polyether surfactant of claim 17,
where the polysiloxane backbone has 24 repeating dimethyl siloxane units and
where the polyethyl ether chain has 16 repeating ethyl ether units.

20. The linear polysiloxane polyether surfactant of claim 17,
where the polysiloxane backbone has 36 repeating dimethyl siloxane units and
where the polyethyl ether chain has 12 repeating ethyl ether units.

21. The linear polysiloxane polyether surfactant of claim 17,
where the polysiloxane backbone has 36 repeating dimethyl siloxane units and
where the polyethyl ether chain has 16 repeating ethyl ether units.

22. The linear polysiloxane polyether surfactant of claim 17,
where the alkyl is propyl.

23. A linear polysiloxane polyether surfactant comprising:
a polysiloxane backbone having 24 repeating dimethyl siloxane units;
a propyl;
a polyethyl ether chain having 12 repeating ethyl ether units; and
a terminating hydroxyl.

24. A linear polysiloxane polyether surfactant comprising:
a polysiloxane backbone having 24 repeating dimethyl siloxane units;
a propyl;
a polyethyl ether chain having 16 repeating ethyl ether units; and
a terminating hydroxyl.

25. A linear polysiloxane polyether surfactant comprising:
a polysiloxane backbone having 36 repeating dimethyl siloxane units;
a propyl;
a polyethyl ether chain having 12 repeating ethyl ether units; and
a terminating hydroxyl.

26. A linear polysiloxane polyether surfactant comprising:
a polysiloxane backbone having 36 repeating dimethyl siloxane units;
a propyl;
a polyethyl ether chain having 16 repeating ethyl ether units; and
a terminating hydroxyl.
